(19) United States
(12) Patent Application Publication
(10) Pub. No.: US 2025/0090172 A1
(43) Pub. Date: Mar. 20, 2025

(54) ELASTIC BAND LIGATION DEVICE

(71) Applicant: BOA MEDICAL, INC., SOLANA BEACH, CA (US)

(72) Inventors: Steve Howard, La Jolla, CA (US); Brad Klos, Solana Beach, CA (US); John Muri, Laguna Niguel, CA (US)

(21) Appl. No.: 18/962,509

(22) Filed: Nov. 27, 2024

Related U.S. Application Data
(63) Continuation-in-part of application No. 18/395,283, filed on Dec. 22, 2023.
(60) Provisional application No. 63/504,367, filed on May 25, 2023.

(51) Int. Cl. A61B 17/12; A61B 17/00

(52) U.S. Cl. CPC A61B 17/12013; A61B 2017/00367; A61B 2017/00477; A61B 2017/00778; A61B 2017/00862; A61B 2017/12018

(57) ABSTRACT

An elastic band ligation device enables a clinician to deploy multiple ligation bands without an assistant and without the need for an external aspirator. A ligation band may be loaded onto a distal end of the ligation device using a detachable loading device, and a pusher may be provided to advance the band from the loading device onto the ligation device. Kits may be provided, for enabling the loading of two or three or more successive ligation bands onto a common ligation device in a single procedure. Methods are also disclosed.

### INCORPORATION BY REFERENCE TO ANY PRIORITY APPLICATIONS

This application is a continuation-in-part of U.S. application Ser. No. 18/395,283, filed Dec. 22, 2023, which claims the priority benefit of U.S. Provisional Patent Application No. 63/504,367, filed May 25, 2023, the entirety of each of which is incorporated by reference here. Any and all applications for which a foreign or domestic priority claim is identified in the Application Data Sheet as filed with the present application are hereby incorporated by reference under 37 CFR 1.57.

### BACKGROUND

### Field

The present disclosure relates to the treatment of hemorrhoids by elastic band ligation. More specifically the disclosure relates to an elastic band ligation device that is provided with an improved elastic band loading assembly.

### Certain Related Art

Treatment of hemorrhoids by elastic band ligation typically involves placing an elastic band on hemorrhoidal tissue in the rectum below the “dentate line” (the junction of the sensitive squamous epithelium known as the “anoderm” and the insensitive lining of the rectum known as the “mucosa”), where there is little sensation. The tissue trapped in the band is cut off from its blood supply, degenerates and is sloughed, and the elastic band along with the sloughed tissue is passed with bowel motions. More importantly, however, the resulting healing process causes the tissue in the vicinity to become fixed, and prolapse of the hemorrhoidal tissue is minimized. This fixation of the tissue serves to eliminate the hemorrhoidal symptoms.

The device disclosed in U.S. Pat. No. 5,741,273 to O'Regan relates to an elastic band ligation device for treatment of hemorrhoids that may be used without directly seeing the site for banding. Thus, it may be used without a proctoscope or anoscope or any other type of scope or viewing technique. Therefore, because the device can be inserted into the rectum and positioned appropriately without simultaneous visualization, or the need for any type of scope, the banding procedure performed in this manner causes less discomfort to the patient. This technique also allows for a more proximal placement of the elastic band, which increases the technique's efficacy as well as causing less discomfort and fewer complications. This device has a suction device incorporated therein to draw hemorrhoidal tissue into an aperture for banding without requiring a second operator or connection to an aspirator.

Ligation is typically performed by initially inspecting the site through a small scope such as a sigmoidoscope, anoscope, proctoscope or other type of scope and making a mental note as to the area where the band should optimally be placed. The scope is then removed and the ligation device is placed in the rectum. This is known as the “Blind” or “Touch” technique.

An alternative application is to visualize the area with an anoscope and insert the banding through the lumen of the anoscope and band directly. This can be done if the operator prefers a direct visual approach. This is known as the “Trans-anoscopic” or “visual” technique.

The distal end of the device is inserted well past the site for banding. Then, the device is gently withdrawn while it is pointed in the direction of the anal canal, minimizing patient discomfort. As the device is being withdrawn, a palpable “ridge” on the outside of the outer tubular member is noted at the anal verge which can be used as a guide to the level where the band should optimally be placed in the rectum. The device is then gently angled or “aimed” toward the hemorrhoid to be treated. In larger patients, the operator may choose to place the device 1-2 cm further in through the anus prior to “aiming” the device towards the hemorrhoid to be treated. In all cases, lubrication is applied liberally onto the device with the loaded elastic band to decrease patient discomfort.

One difficulty with the foregoing type of device involves loading the elastic band onto the ligation device. This is optimally done at the clinical site, since pre-loading the band at the point of manufacture will cause the stretched band to lose elasticity. At the clinical site, the small elastic band of the O'Regan type device must be manually pushed up the cone shaped loading device to reach the distal end of the ligation device. This motion causes the band to expand to the desired diameter to fit the end of the O'Regan device. However, pushing the small elastic band up the loading cone must be done by a gloved physician or assistant, in a slippery environment due to the device lubrication, and can be difficult and time consuming to achieve.

### SUMMARY

A single operator elastic band ligation device is provided that is self-contained having all the necessary elements to complete the ligation of internal hemorrhoidal tissue, including the capability of suctioning the hemorrhoidal tissue into a cylinder, and the placing of an elastic band around the tissue. The elastic band is expanded and positioned around the front end of an inner tubular member. The band is discharged by advancing an outer sleeve over the inner tubular member and thus placing the elastic band over the hemorrhoidal tissue sucked into the inner tubular member.

In one embodiment, the ligation device is disposable after use.

In one implementation of the invention, there is provided a pusher for loading an elastic band on a ligation device. The pusher comprises a base; at least two arms extending in a proximal direction from the base, the arms inclined radially inwardly in the proximal direction and defining a proximal opening; the arms movable between a first position which defines a first diameter for the opening and a second, radially outwardly displaced position, which defines a second, larger diameter opening in response to distal advance of a loading device therein.

The pusher may further comprise an elastic band carried by the arms to bias the arms in the direction of the first position.

In some embodiments, the pusher may have arms of predetermined and variable stiffness that bias the radial compression of the arms in the first position.

In some embodiments, the pusher arms will have features that engage the band in a way to promote radial expansion as the device advances in the axial direction.

In some embodiments, the engagement features prevent the elastic bands from rolling and twisting and promote true radial expansion before the band is released.

The pusher may comprise two or three or four or more arms. The base may comprise a radially outwardly extending projection such as an annular flange. The arms may be integrally formed with the base. A ligation band engagement structure may be provided on the distal ends of the arms, for rolling the ligation band along the loading device. The ligation band engagement structure may comprise a distally facing ramp.

The base may be provided with an aperture, in communication with the proximal opening through a central lumen defined by the arms. The aperture may be provided with a rotational alignment feature configured to engage a complementary alignment feature on the loading device. The alignment feature may comprise a keyway such as a radially inwardly extending projection or a radially outwardly extending recess formed in the base adjacent the aperture.

There is also provided a loading device, configured for loading an elastic band onto a ligation device. The loading device comprises a body having a proximal end and a distal end, the body increasing in diameter from the distal end to the proximal end; an engagement structure carried by the proximal end, configured for releasable engagement with a distal end of a ligation device; and an axially extending guide along the surface of the body, for rotational engagement with an elastic band pusher while permitting axial relative movement between the loading device and the pusher.

The engagement structure may comprise a proximal projection configured to be removably received within a distal aperture on the ligation device. The loading device may additionally comprise a ligation band carried proximate the distal end of the loading device.

There is also provided a ligation device loading kit including a pusher, a loading device, and at least one ligation band. The ligation band may be preloaded on the loading device. The ligation device loading kit may additionally comprise a ligation device, configured to detachably connect to the loading device. The kit may comprise at least three loading devices and at least three ligation bands.

There is also provided a method of loading a ligation band onto a ligation device. The method comprises the steps of removably attaching a proximal end of a loading device to a distal end of the ligation device, the loading device carrying a ligation band; positioning a distal end of the loading device into a proximal end of a pusher; and advancing the ligation device distally relative to the pusher, causing the pusher to advance the ligation band proximally along the loading device and onto the ligation device. The proximal end of the pusher may be provided with a ligation band engagement structure which causes the ligation band to roll along the loading device during the advancing step. The pusher may also be configured to prevent rolling and only perform radial expansion.

In some embodiments, the system may include multiple loading devices to facility ease of use for the soiled or lubricated device.

In some embodiments, the pusher and loading frame may have features to prevent re-use.

In some embodiments, the elastic bands can contain latex or can be bands that are latex free.

In some embodiments, the bands can be stored on the loading device in a configuration to maximize their shelf life stability. The range of pre-expansion can range from 5%-50% with the optimal stretch being 25%.

In some embodiments, the devices could be packaged and configured to be delivered in a sterile or non-sterile configuration

In some embodiments, the bands and loading devices could be configured to be disposable and the delivery device could be separately packaged.

In some embodiments, the bands and components can be color coded to identify the presence of latex.

In some embodiments, the expanded bands can be packaged in a moisture resistant packaging system to prevent degradation to the elastic band.

In some embodiments, the expanded bands can be packaged in a UV resistant package to prevent exposure and UV degradation to the elastic band.

In some embodiments, the expanded bands can be packaged in a vacuum sealed, inert gas purged package to prevent oxidation and degradation to the band,

There is also provided a method of treating a patient. The method comprises providing a ligation device that has previously deployed a first ligation band into a patient; attaching a loading device to a distal end of the ligation device; advancing a second ligation band distally along the loading device by advancing the loading device distally with respect to a pusher such that the pusher advances the second ligation band along the loading device and onto the ligation device; removing the loading device from the ligation device; and deploying the second ligation band into the patient.

The disposable elastic band ligation device may be used in a doctor's office and does not generally require any form of anesthetic. One to three bands are generally placed at each patient visit and as many as six bands may be required in total, particularly in advanced cases. In one embodiment the ligation device is made of plastic and is disposable, therefore, once it has been used it is discarded and does not have to be sterilized. In another embodiment, the ligation device is made of metal and may be sterilized after each use.

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1 is a sectional elevational view showing an elastic band ligation device according to one embodiment of the present disclosure,

FIG. 2 is an end view showing the ligation device of FIG. 1,

FIG. 3 is a partial sectional elevational view showing a cone shaped loading device for expanding an elastic band as it is loaded onto the ligation device,

FIG. 4 is a sectional elevational view showing an elastic band ligation device similar to that shown in FIG. 1 including an obturator.

FIG. 5 is a partial elevational view showing a portion of an inner tubular member of an elastic band ligation device with an angled front end.

FIG. 6 is a perspective view of one implementation of the ligation device.

FIG. 7 is a perspective view of one implementation of a loading device for loading an elastic band onto the ligation device of FIG. 6.

FIGS. 8A-8B are perspective views of an elastic band pusher.

FIGS. 9A and 9B are perspective views of the pusher of FIGS. 8A-8B in position over a distal tip of the loading device.

FIG. 10 schematically illustrates a kit having a pusher and a plurality of loading devices which may each be pre-loaded with an elastic band in a small diameter configuration.

FIG. 11 is a perspective view showing an elastic band ligation device according to another embodiment of the present disclosure.

FIG. 12 is a sectional perspective view showing an elastic band ligation device of FIG. 11.

FIG. 13 is a perspective view showing an elastic band ligation device according to another embodiment of the present disclosure.

FIG. 14 is a sectional perspective view showing an elastic band ligation device of FIG. 13.

FIG. 15 is a partially exploded perspective view showing an elastic band ligation device according to another embodiment of the present disclosure;

FIG. 16 is another perspective view of an elastic band ligation device according to another embodiment of the present disclosure;

FIG. 17 is a sectional view showing an elastic band ligation device of FIG. 16;

FIG. 18 is a perspective view of an elastic band pusher according to another embodiment of the present disclosure;

FIG. 19 illustrates ligation band loading cone according to one embodiment of the present disclosure;

FIG. 20 illustrates ligation band loading stand according to one embodiment of the present disclosure;

FIG. 21 illustrates ligation band loading process and interface according to one embodiment of the present disclosure;

FIG. 22 illustrates cross section and detail of ligation band loading process and interface according to one embodiment of the present disclosure.

FIG. 23 is another perspective view of an elastic band ligation device according to another embodiment of the present disclosure;

FIG. 24 is another perspective view of an elastic band ligation device component according to another embodiment of the present disclosure;

FIG. 25 is another perspective view of a complete elastic band ligation device system according to another embodiment of the present disclosure;

FIG. 26 is another perspective view of a complete elastic band ligation device system according to another embodiment of the present disclosure;

FIG. 27 is another perspective view of a partially exploded elastic band ligation device system according to another embodiment of the present disclosure;

FIG. 28 is another perspective view of a exploded elastic band ligation device system according to another embodiment of the present disclosure;

FIG. 29 is a side view of an elastic band ligation device with an identified cross sectional cut location according to another embodiment of the present disclosure;

FIG. 30 is a sectional view from FIG. 29.

FIG. 31 is a close-up of the sectional view in FIG. 30.

### DETAILED DESCRIPTION

In one embodiment, the elastic band ligation device of the present disclosure is made out of plastic, either clear or opaque plastic, and is disposable. In another embodiment, the ligation device is made from stainless steel and may be sterilized after each use. As shown in FIG. 1, the ligation device 10 has an inner tubular member 12 with a rounded, beveled or radiused front end 14 which has an opening 15 slightly smaller in diameter than the inner tubular member 12. The inner tubular member 12 may be slightly tapered increasing in diameter as it extends back from the front end 14. In another embodiment the inner tubular member 12 may be cylindrical in shape with no taper. The length of the inner tubular member 12 is sufficient for insertion into the rectum of a patient for treatment of hemorrhoidal tissue. The inner tubular member 12 is integral with a plunger housing 16 which has a plunger 18 therein, thus when the plunger 18 is moved back in the plunger housing 16, a suction occurs at the opening 15 of the front end 14. In another embodiment, the plunger housing 16 and inner tubular member 12 may be separate components joined together.

A stretched elastic band 20, or rubber band as referred to in some cases, is positioned over the inner tubular member 12 adjacent the front end 14. An outer tubular pusher sleeve 22 fits over the inner tubular member 12 and has a limited friction fit on the inner tubular member 12. When the outer tubular pusher sleeve 22 is pushed forwards toward the front end 14 of the inner tubular member 12, the elastic band is pushed off the front end 14 of the inner tubular member 12 and engages hemorrhoidal tissue which has been sucked into the opening 15 in the front end of the inner tubular member 12.

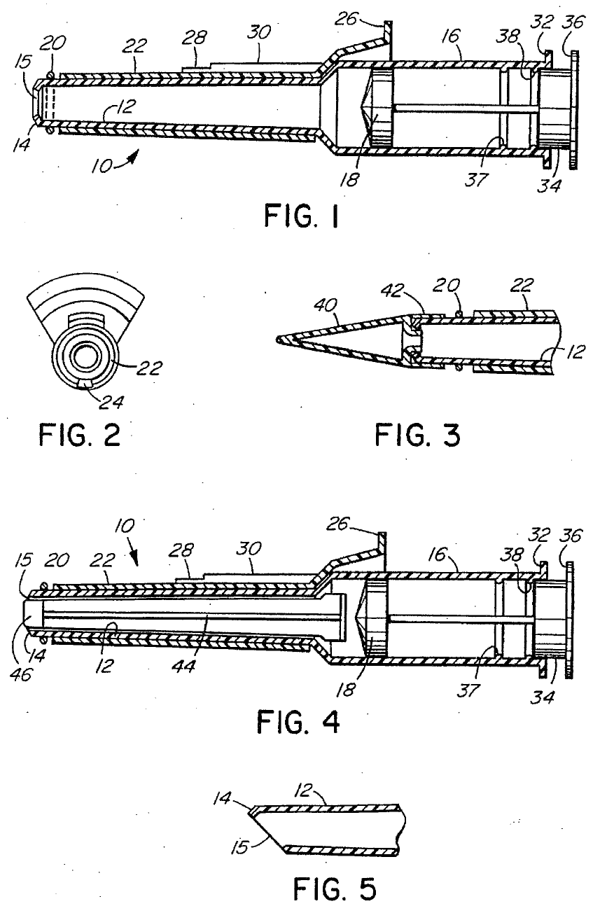

The outer tubular pusher sleeve 22 is shown having a longitudinal slit 24 as shown in FIG. 2 so the sleeve is in effect a clip around the inner tubular member 12. In another embodiment, the pusher sleeve 22 is tubular and slides over the inner tubular member 12. On one side of the outer tubular pusher sleeve 22 is a partial pusher flange 26 extending from the end of the pusher sleeve 22. The pusher flange 26 allows the pusher sleeve 22 to be pushed by a thumb of an operator so the elastic band 20 is discharged from the front end 14 of the inner tubular member 12. A first raised step 28 and a second raised step 30 are shown on one side of the pusher sleeve 22 adjacent the pusher flange 26. These steps are provided for an operator to position the ligation device 10 either by sight or by feel. In one embodiment, longitudinal ribs (not shown) are provided on the inside of the pusher sleeve 22 for friction purposes to ensure the pusher sleeve does not slide freely on the inner tubular member 12 and only moves when pushed by an operator.

The plunger housing 16 is substantially cylindrical and is not tapered and an outside shoulder 32 allows the housing 16 to be gripped when the plunger 18 is pulled back. The plunger 18 is attached to a stem 34 having a cross-section as shown in FIG. 1, with a shoulder 36 to act as a grip so that the plunger 18 can be pulled out of the housing 16 to draw suction through the opening 15 in the front end 14 of the inner tubular member 12.

A first shoulder ring 37 is located towards the end of the housing 16 and acts as a locking system so that when the plunger 18 is pulled back and rides over the first shoulder ring 37 it is held in that position and holds a vacuum in the housing 16 and the inner tubular member 12. If there was no locking system, the plunger 18 would immediately return to the original position and no vacuum would remain to pull the hemorrhoidal tissue into the inner tubular member 12. A second shoulder ring 38 is positioned outside the first shoulder ring 37 and acts as a stop to prevent the plunger 18 from being pulled out of the housing 16.

Whereas shoulders 37, 38 are shown for locking the plunger 18 in the housing 16, it will be apparent to those skilled in the art that other types of clamping systems to stop the plunger 18 returning in the housing 16 may be provided.

In operation once a doctor has examined the rectum of a patient and discovered the position of the hemorrhoidal tissue, he removes the scope through which the inspection has initially occurred, memorizing the position of the hemorrhoidal tissue and then inserts the ligation device similar to that shown in FIG. 1. The opening 15 at the front end 14 of the inner tubular member 12 is positioned as close as possible to the hemorrhoidal tissue and the operator holds the housing 16, draws back the plunger 18 past the first shoulder ring 37 so a vacuum is formed in the housing 16 and the hemorrhoidal tissue is pulled into the inner tubular member 12 through the opening 15. When this has been accomplished, the doctor releases the shoulder 36 of the plunger 18 and pushes the outer plastic pusher sleeve 22 forward so that the stretched elastic band 20 is pushed off the front end 14 of the inner tubular member 12 to capture the hemorrhoidal tissue. The ligation device can then be removed and the procedure may be carried out at least three times during one patient's visit to the doctor's office. No aspirator or other device need be attached to the ligation device as the built in plunger mechanism sucks the hemorrhoidal tissue into the inner tubular member 12 and holds it there for the doctor to place the elastic bands.

A cone shaped loading device 40 is shown in FIG. 3 and has a thin sleeve 42 that overlaps the front end 14 of the inner tubular member 12. An elastic band 20 is pushed over the loading device 40 and expanded as it is moved along until the elastic band 20 is pushed off the sleeve 42 onto the front end 14 of the inner tubular member 12. The loading device 40 is then removed and the elastic band is moved to the position shown in FIG. 1.

The thin sleeve 42 of the loading device 40 prevents the elastic band becoming lodged in the join between the loading device 40 and the inner tubular member 12.

In another embodiment an obturator 44 is provided which fits inside the inner tubular member 12 as shown in FIG. 4. The obturator 44 has a button shaped front end 46 which protrudes slightly through the opening 15 at the front end 14 of the inner tubular member 12. The purpose of the obturator 44 is to improve access of the ligation device 10 when it is inserted into the rectum. It avoids the edge of the opening 15 at the front end 14 from snagging on hemorrhoidal tissue thus causing discomfort to a patient. The plunger 18 resting and pushing against the outer end of the obturator 44, maintains it in position as the device is being inserted into the rectum. Once the ligation device 10 has been positioned, the doctor draws back the plunger 18 and the obturator 44 moves back with the plunger 18 exposing the opening 15 at the front end 14 of the inner tubular member 12 and hemorrhoidal tissue is pulled into the inner tubular member 12 through the opening 15.

As shown in FIG. 1, the front (distal) end 14 of the inner tubular member 12 is rounded or radiused to allow a doctor to insert the ligation device 10 at any angle and not cause snagging or discomfort to a patient. The ligation devices 10 may be made in different sizes. 8 mm, 10 mm and 12 mm inside diameters of the inner tubular member 12 are preferred sizes. The dimension of the opening 15 is generally about 0.5 to 1 mm less than the internal diameter of the inner tubular member to permit radiused rounded edges at the front end 14. The obturator 44 is generally used in the larger ligation devices although may be provided in all sizes.

In another embodiment, shown in FIG. 4, the front end 14 of the inner tubular member is at an angle and not perpendicular to the axis of the inner tubular member 12. This angled front end 14 and opening 12 is for ease of placing the ligation device on the side wall of the rectum to capture hemorrhoidal tissue.

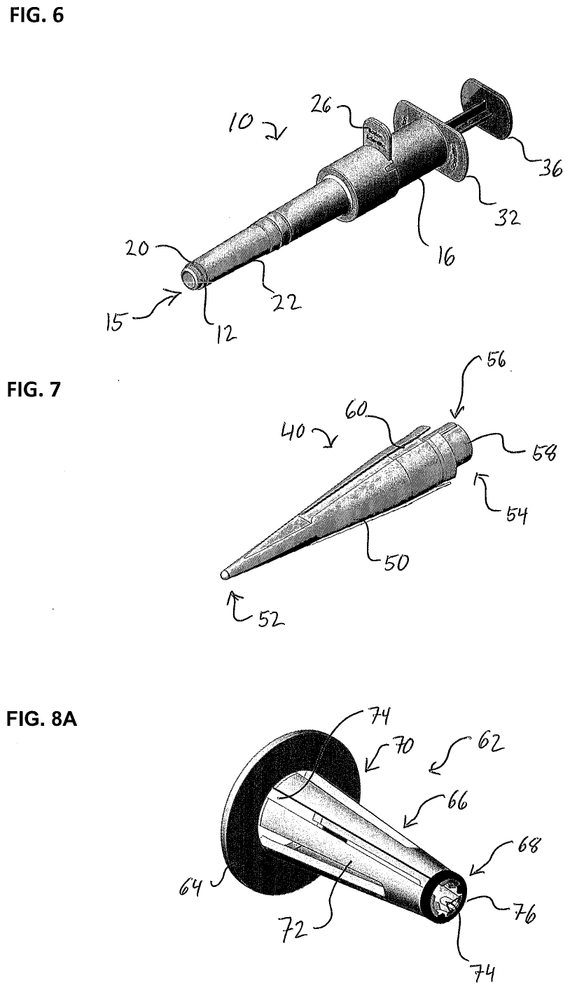

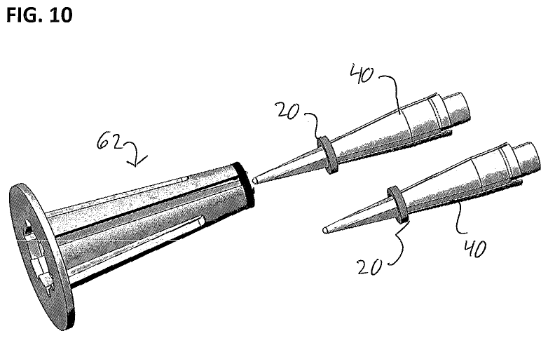

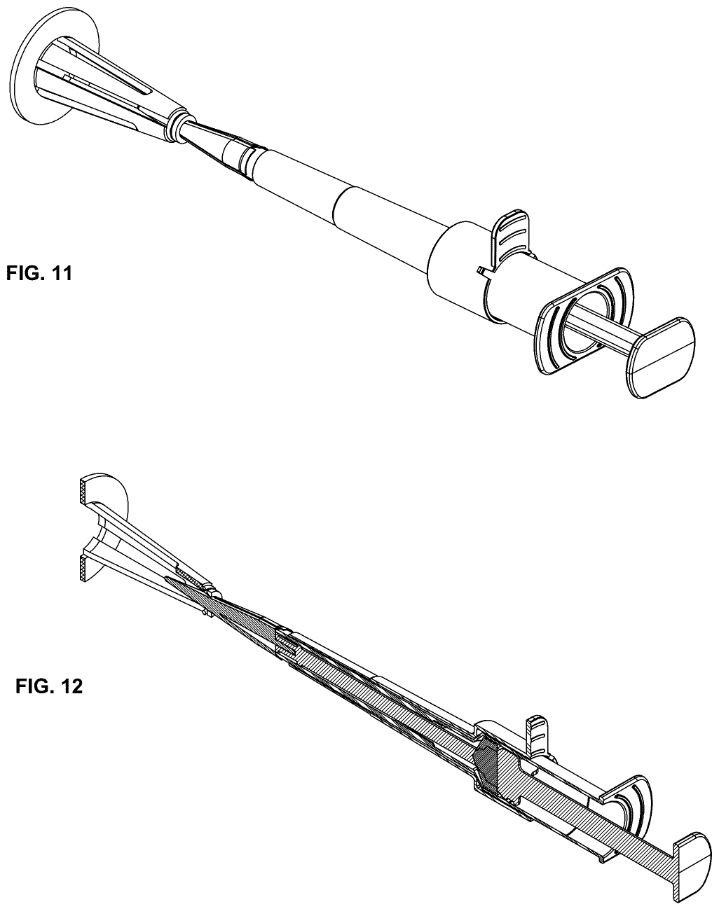

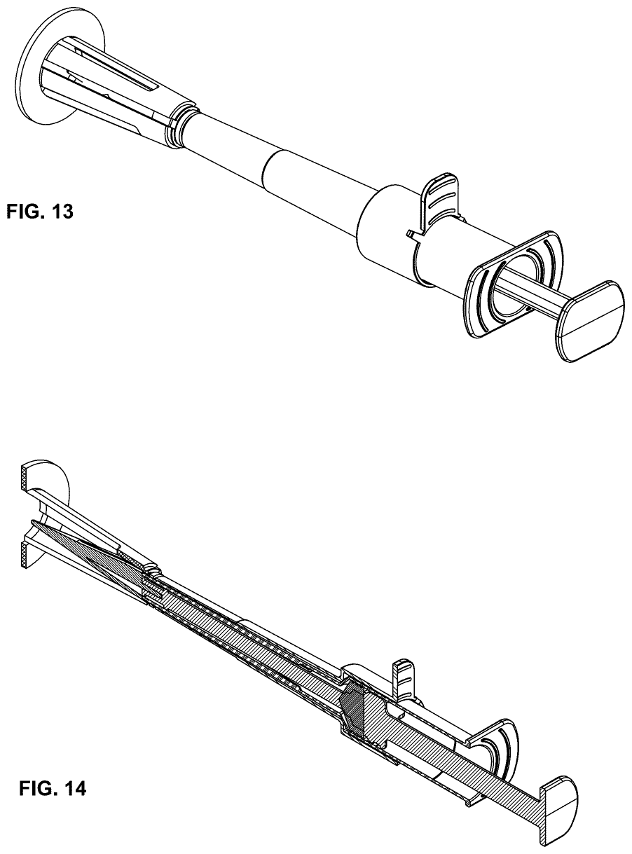

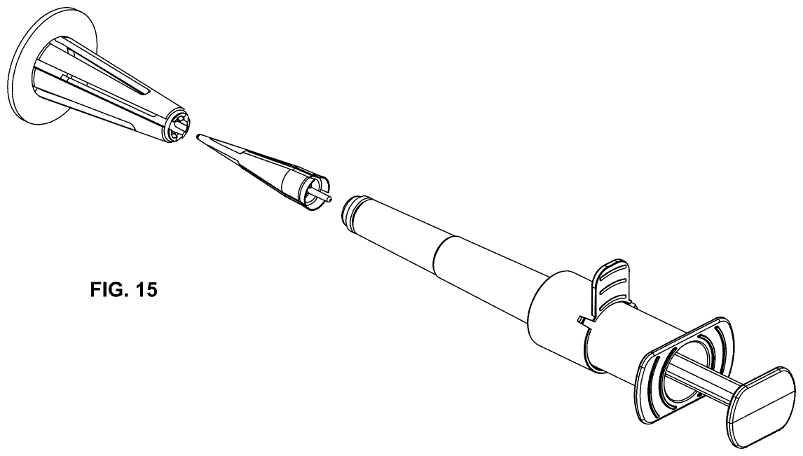

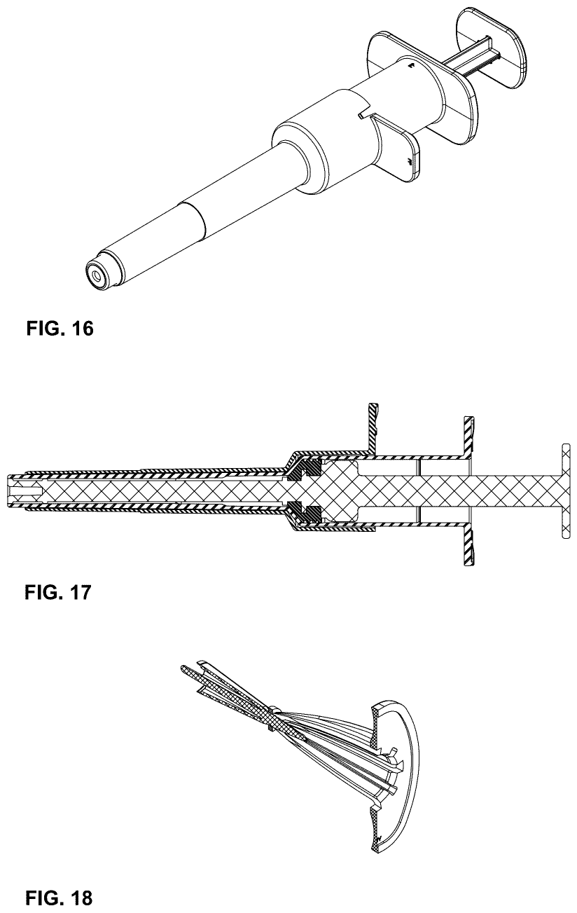

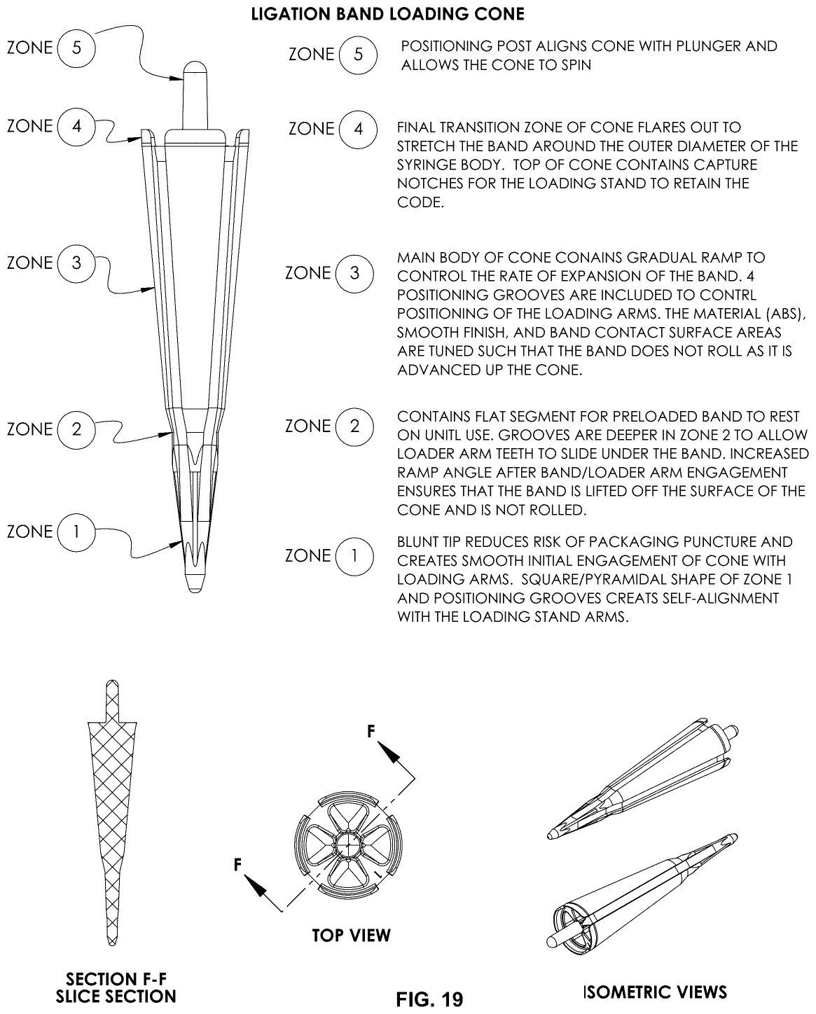

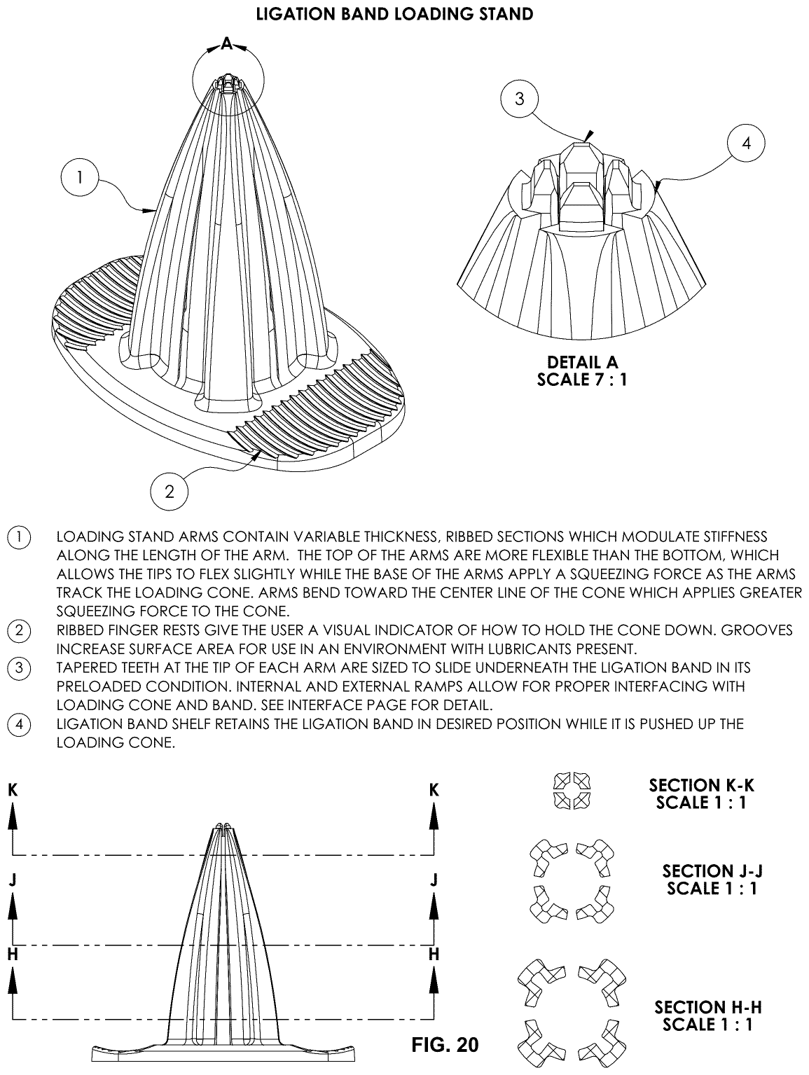

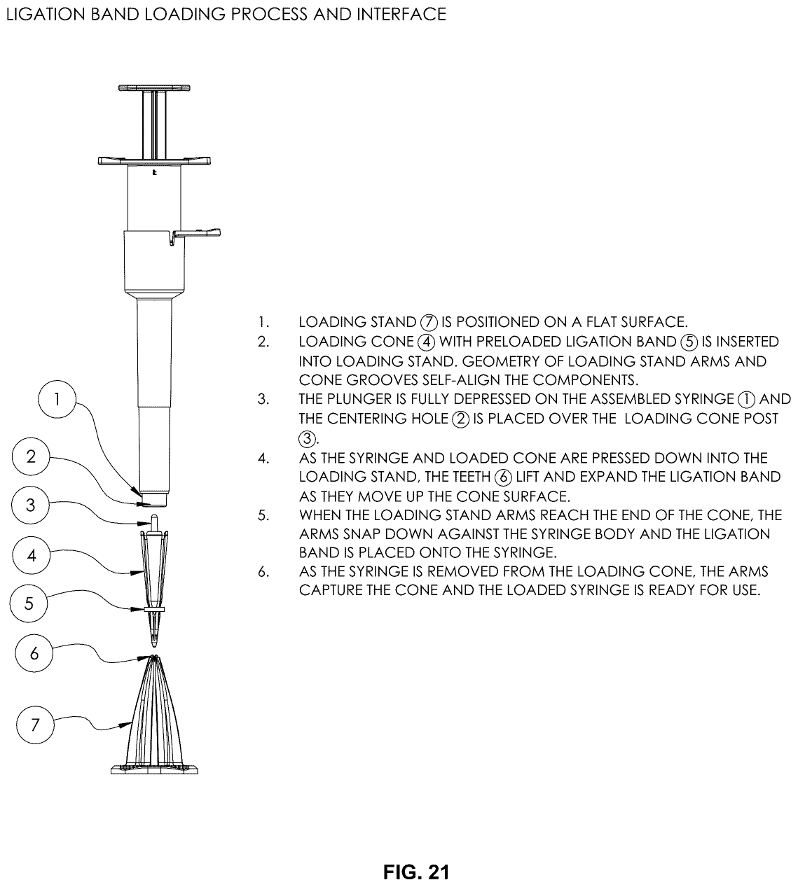

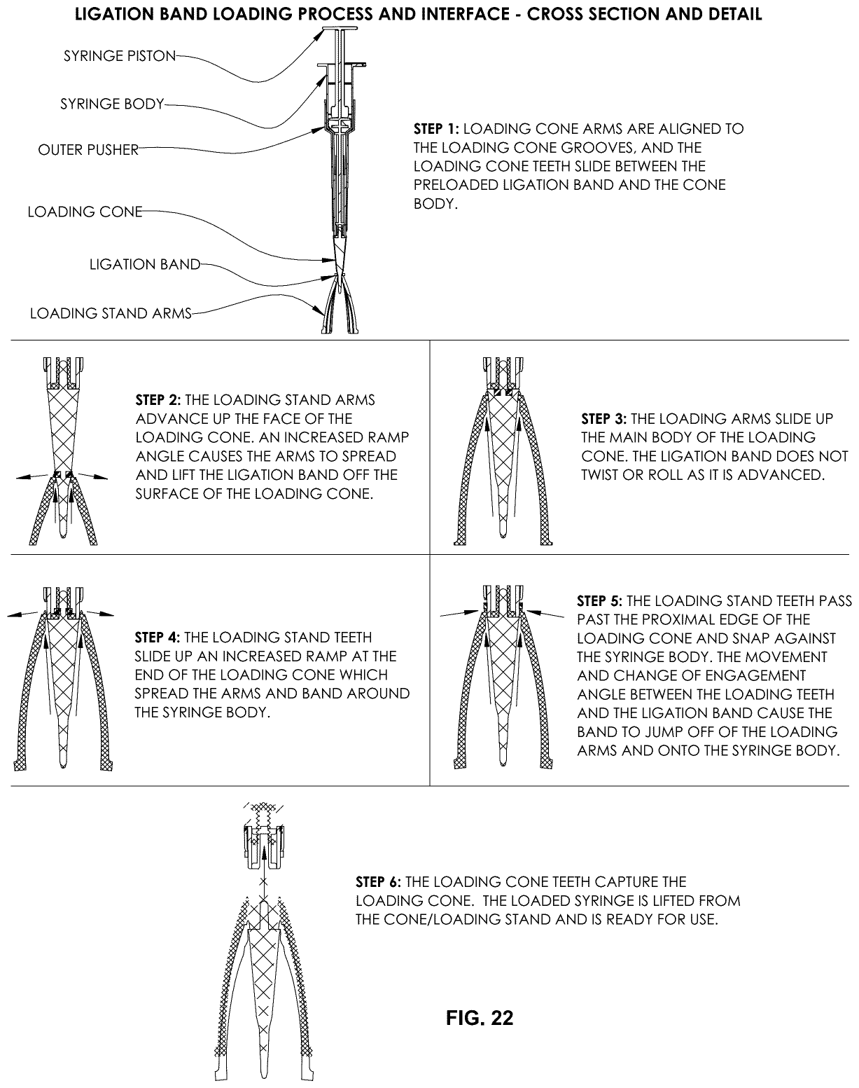

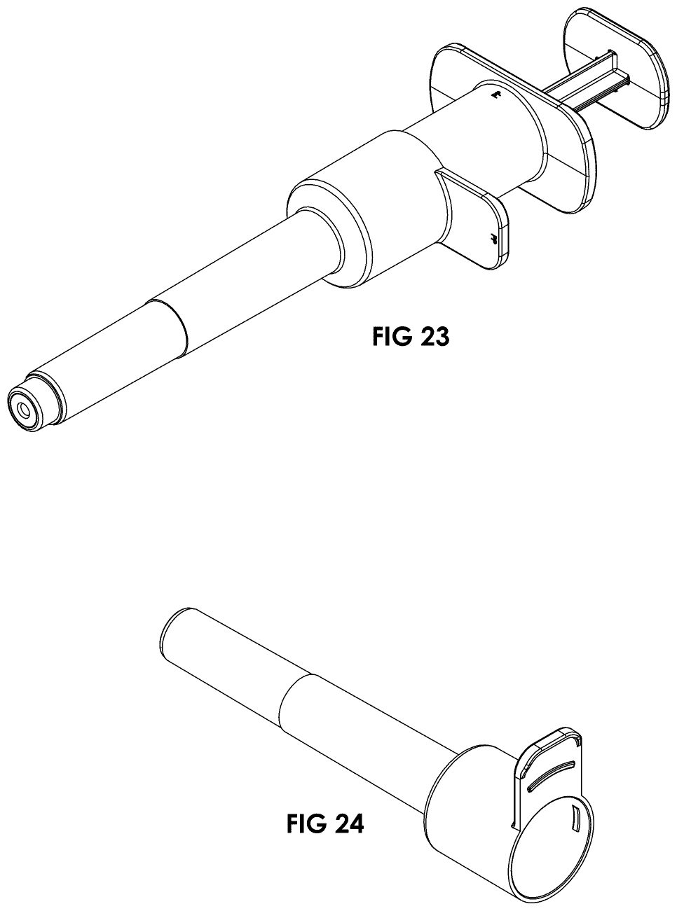

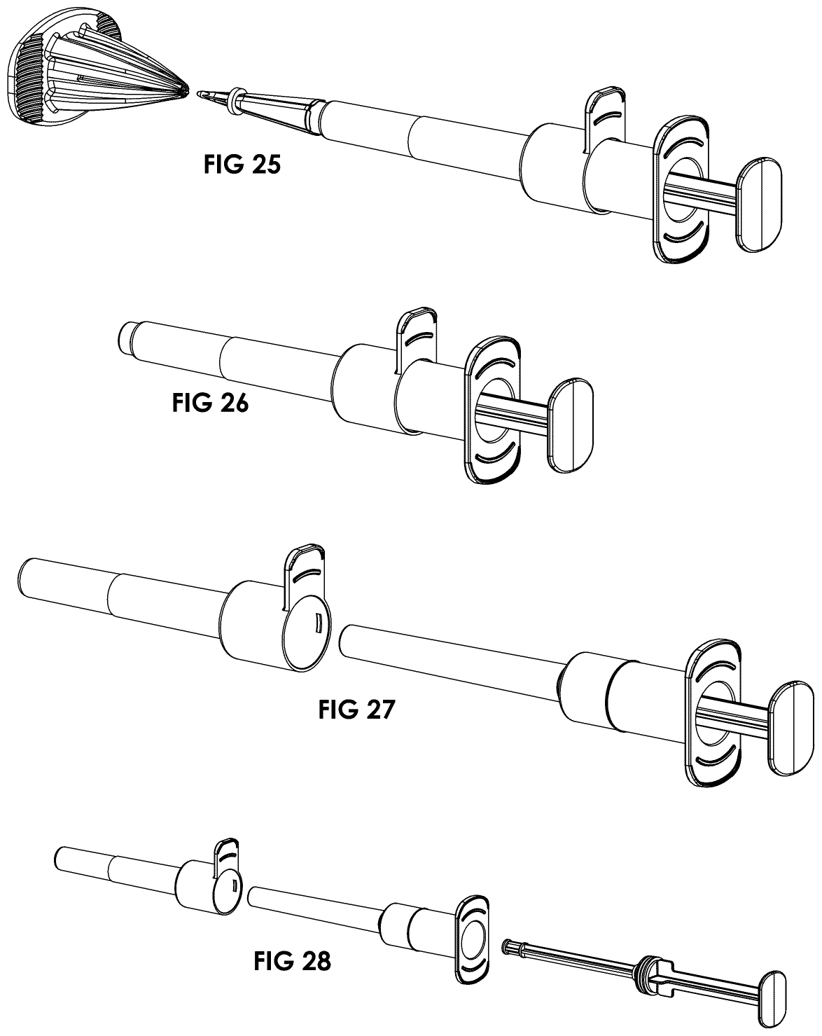

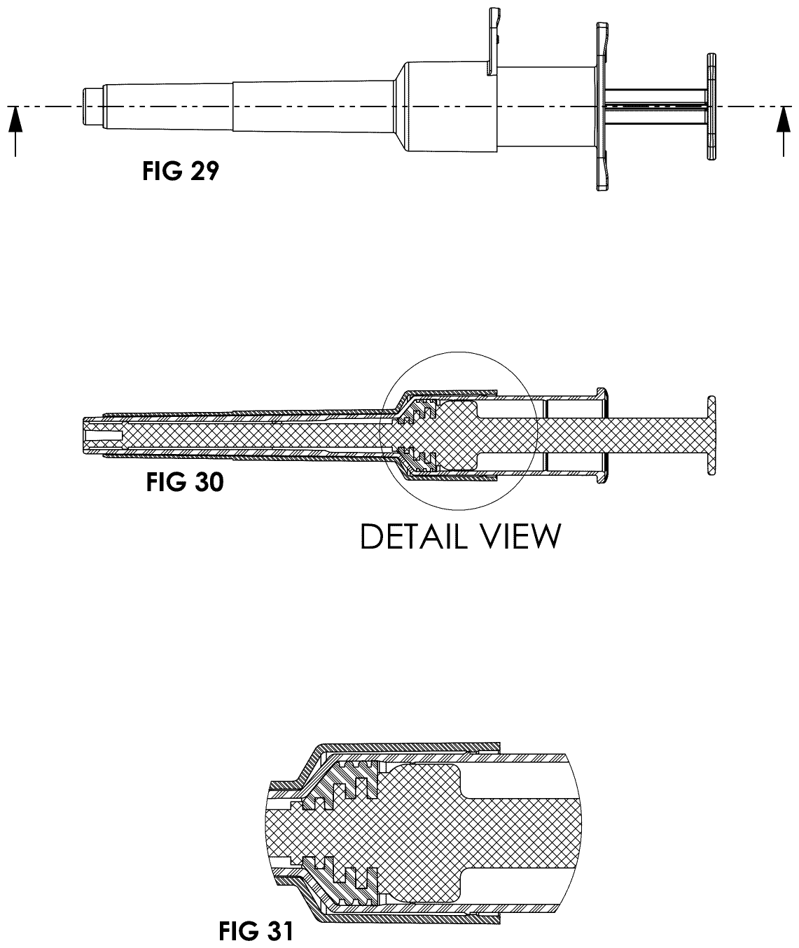

A ligation band loading device in accordance with the present invention is discussed in connection with FIGS. 6 through 10. A loading device 40 (FIG. 7) is configured for a removable attachment to the distal (front) end 14 of the ligation device 10. The loading device 40 comprises an elongate ramped or conical body 50 which increases in outside diameter from the distal end 52 to the proximal end 54. Proximal end 54 may be provided with a ligation device engagement structure 56 such as an axially proximally extending projection 58 for removable insertion into the distal opening 15 on ligation device 10. Loading device 40 is configured to allow an elastic band to be advanced in a proximal direction along the ramped surface, progressively radially expanding the elastic band until it has advanced proximally beyond the proximal end 54 of the loading cone 40 and onto the landing zone on the distal end of the inner tubular member 12.

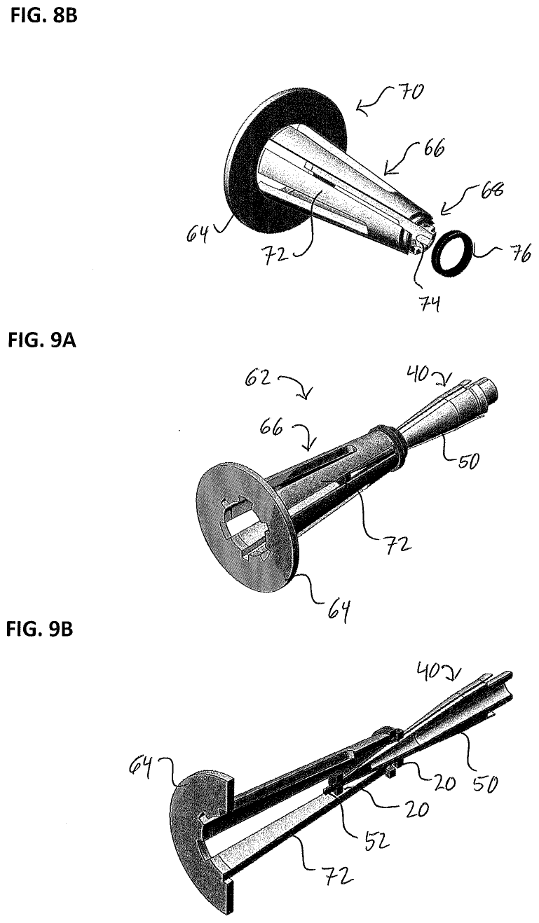

To facilitate proximal advance of the elastic band 20 along the cone 40 and up onto the inner tubular member 12, there is provided a pusher 62, one implementation of which is illustrated in FIGS. 8A-8B. The pusher 62 comprises a base 64, carrying a loading frame 66 extending between a distal end 70 attached to the base 64 and a proximal end 68. The loading frame 66 comprises at least one and preferably two or three or four or more arms 72 which incline radially inwardly in the proximal direction. Arms 72 surround a central lumen which extends throughout the length of the loading frame 66 to a proximal end opening 74. The proximal ends of arms 72 are radially outwardly movable between a reduced diameter configuration as in FIGS. 8A and 8B, and a radially enlarged diameter configuration as will be discussed. Arms 72 may be pivotably secured to the base 64, and/or may comprise a flexible material such as any of a variety of medical grade polymers. Preferably, the arms 72 are biased into the reduced diameter configuration, and an elastic retention band 76 may be carried by the arms 72 to provide an elevated radially inwardly directed bias.

In use, the loading device 40 is coupled to the distal end of the ligation device 10, such as by advancing the axial proximal extension 58 into the opening 15 on the ligation device 10 (See FIGS. 9A and 9B). An elastic ligation band 20 is carried on the distal end of the conical body 50 of the loading device 40.

The distal end 52 of the loading device 40 is positioned within the proximal end 68 of the pusher 62 such that the elastic band 20 carried by the conical body, 50 is in contact with the proximal ends 68 of the loading frame 66.

The loading device 40 is there after axially advanced into the loading frame 66, deflecting the arms 72 radially outwardly to accommodate the progressively increasing diameter of the conical body 50 extending therein. This may be accomplished by placing the base 64 against a table or other surface and pushing the ligation device in a distal direction. The distal face of the arms 72 advance the elastic band proximally up the ramped surface of the conical body 50, until the elastic band is deposited on the outside surface of the inner tube member 12. The pusher 62 and loading device 40 may thereafter be detached from the ligation device 10, which is now ready for a ligation procedure.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise. Additionally, as used herein, “gradually” has its ordinary meaning (e.g., differs from a non-continuous, such as a step-like, change).

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A pusher for loading an elastic band on a ligation device, comprising:
a base;
at least two arms extending in a proximal direction from the base, the arms inclined radially inwardly in the proximal direction and defining a proximal opening; and
the arms movable between a first position which defines a first diameter for the opening and a second, radially outwardly displaced position, which defines a second, larger diameter opening in response to distal advance of a loading device therein.

2. A pusher as in claim 1, further comprising an elastic band carried by the arms to bias the arms in the direction of the first position.

3. A pusher as in claim 1, comprising three arms.

4. A pusher as in claim 1, wherein the base comprises a radially outwardly extending flange.

5. A pusher as in claim 4, wherein the arms are integrally formed with the base.

6. A pusher as in claim 3, further comprising an aperture in the base, in communication with the proximal opening through a central lumen defined by the arms.

7. A pusher as in claim 6, comprising a rotational alignment feature configured to engage a complementary alignment feature on the loading device.

8. A pusher as in claim 7 wherein the alignment feature comprises a keyway formed in the base adjacent the aperture.

9. A pusher as in claim 7 wherein the alignment feature comprises a projection formed in the base adjacent the aperture.

10. A pusher as in claim 3, further comprising a ligation band engagement structure on distal ends of the arms, for rolling the ligation band along the loading device.

11. A pusher as in claim 10, wherein the ligation band engagement structure comprises a distally facing ramp.

12. A loading device, configured for loading an elastic band onto a ligation device, comprising:
a body having a proximal end and a distal end, the body increasing in diameter from the distal end to the proximal end;
an engagement structure carried by the proximal end, configured for releasable engagement with a distal end of a ligation device;
an axially extending guide along a surface of the body, for rotational engagement with an elastic band pusher while permitting axial relative movement between the loading device and the pusher; and
wherein the loading device can control an exact radial expansion of the elastic band to a prescribed percentage;
wherein the loading device can be radially larger on an axial axis;
wherein the loading device can be isolated from environmental conditions.

13. A loading device as in claim 12, wherein the engagement structure comprises a proximal projection configured to be removably received within a distal aperture on the ligation device.

14. A loading device as in claim 12, further comprising a ligation band carried proximate the distal end.

15. A ligation device loading kit comprising a pusher as in claim 1, a loading device as in claim 12 and at least one ligation band.

16. A ligation device loading kit as in claim 15, wherein the ligation band is carried by the loading device.

17. A ligation device loading kit as in claim 15, further comprising a ligation device, configured to detachably connect to the loading device.

18. A ligation device loading kit as in claim 15, comprising at least three loading devices and at least three ligation bands.

19. A method of loading a ligation band onto a ligation device, comprising steps of:
removably attaching a proximal end of a loading device to a distal end of the ligation device, the loading device carrying a ligation band;
positioning a distal end of the loading device into a proximal end of a pusher; and
advancing the ligation device distally relative to the pusher, causing the pusher to advance the ligation band proximally along the loading device and onto the ligation device.

20. A method of loading a ligation band as in claim 19, wherein a ligation band engagement structure on a proximal end of the pusher causes the ligation band to roll along the loading device during the advancing step.

21. A method of treating a patient, comprising steps of:
providing a ligation device that has previously deployed a first ligation band into a patient;
attaching a loading device to a distal end of the ligation device;
advancing a second ligation band distally along the loading device by advancing the loading device distally with respect to a pusher such that the pusher advances the second ligation band along the loading device and onto the ligation device;
removing the loading device from the ligation device; and
deploying the second ligation band into the patient.
